(19) United States
(12) Patent Application Publication
(10) Pub. No.: US 2022/0257625 A1
(43) Pub. Date: Aug. 18, 2022

(54) COMPOSITION AND METHODS FOR THE TREATMENT OF SKIN CONDITIONS

(71) Applicant: BIODERM, INC., Madison, WI (US)

(72) Inventor: Lorraine Faxon Meisner, Madison, WI (US)

(21) Appl. No.: 17/627,474

(22) PCT Filed: Jul. 15, 2020
(86) PCT No.: PCT/US20/42150

§ 371 Date: Jan. 14, 2022

Related U.S. Application Data
(60) Provisional application No. 62/874,265, filed on Jul. 15, 2019.

(51) Int. Cl. A61K 31/7008; A61K 9/00; A61K 33/00; A61K 31/375; A61K 31/05; A61P 17/02; A61P 17/10; A61K 31/198; A61K 47/46

(52) U.S. Cl. CPC A61K 31/7008; A61K 9/0014; A61K 9/0063; A61K 33/00; A61K 47/46; A61K 31/05; A61P 17/02; A61P 17/10; A61K 31/198; A61K 31/375

(57) ABSTRACT

The present disclosure provides compositions and methods of use thereof for the treatment of skin diseases and disorders or periodontal disease. The compositions comprise cannabidiol, derivatives or pharmaceutical salts thereof, and ascorbic acid. The formulations may also include an anti-inflammatory agent, such as glucosamine.

### CROSS-REFERENCE TO RELATED APPLICATIONS

This application claims the benefit of U.S. Provisional Application No. 62/874,265, titled Jul. 15, 2019, the contents of which is incorporated herein by reference.

### FIELD

The present disclosure provides novel compositions and methods for treatment of skin or periodontal diseases and disorders. More particularly the current invention pertains to a composition comprising ascorbic acid and cannabidiol (CBD) or derivatives thereof.

### BACKGROUND

Acne rosacea is a chronic inflammatory skin condition affecting the face and eyelids of certain middle-aged adults. Clinical signs include erythema (redness), dryness, papules, pustules, and nodules either singly or in combination in the involved skin areas. In the classic situation, the condition develops in adults between the ages of 30 and 50. While certain lesions of acne rosacea may mimic acne lesions, the processes are separate and distinct. The etiology of acne rosacea has been a frequently-discussed but little consensus has ever been reached. However, treatment with medications to block such vasomotor flushing have no effect on other aspects of the disease such as papules and pustules. Treatment with oral antibiotics has been shown to effectively block progression of rosacea through a poorly-understood anti-inflammatory mechanism. A few cases in which a topical formulation of ascorbic acid, zinc and tyrosine was applied to acne rosacea, for example, a slight improvement was observed with long-term use. Such formulations, however, have not been shown to clear up acne rosacea to a substantial extent and even slight improvement requires long-term, diligent daily application to the affected skin. Improved formulations of topical ascorbic acid are needed to effectively and quickly treat skin maladies such acne rosacea, allergic inflammations and hypersensitivity.

Acne is a complex skin disease comprising inflammation triggered by various processes such as seborrhea, hormonal imbalances, immune reactions and infectious and environmental factors. Acne can also occur when skin cells plug hair follicles. In westernized societies, acne is a nearly universal skin disease afflicting 79% to 95% of the adolescent population. In men and women older than 25 years. 40 to 54% have some degree of facial acne, and clinical facial acne persists into middle age in 12% of women and 3% of men. There are differences in certain properties of the skin, such as the density and the number of layers in the stratum corneum (SC), with a thicker and more compact SC in African Americans and a much thinner SC in Asian skin, but acne treatment is similar in all races. Although acne lesions may appear similar in skin of color, dark skinned individuals can develop inflammatory papules, nodules and cysts that lead to post inflammatory hyperpigmentation (NH). Also, acne scars can lead to keloid formation or hypertrophic scarring in addition to PIH in Fitzpatrick skins Type IV to VI. Topical retinoid therapy has been a major part of the foundation of therapy for acne since the availability of topical tretinoin. However, there are well known and documents side effects and adverse reactions in many individuals. For those suffering from persistent, severe acne, few medical-grade treatment options exist. Retinoid isotretinoin (e.g. Accutane) is often a last resort due to its lengthy list of side effects. This prescription treatment attacks the sebaceous glands in an effort to reduce oil production. It is not only hard on the skin, but it is also hard on the body.

The UV spectrum that reaches the earth's surface contains UVB (280-320 nm) and UVA (320-400 nm) resulting in different levels and types of protein damage. U.S. FDA conducted a study that found skin and blood supply absorption of four ingredients in sunscreens, predominantly oxybenzone which is also present in many personal care products. Increased levels of oxybenzone were evident after the first day, and increased from day one to day four, suggesting accumulation. Oxybenzone has already been found in human breast milk, amniotic fluid, blood, and urine, and since most of the sunscreen is washed off, it has accumulated in waterways of the ecosystem where it can affect fish, coral, and other aquatic species. Other sunscreen ingredients have also been detected in blood and human breast milk, but much less of each is absorbed compared to oxybenzone. In 2019 and 2020, the FDA published two follow-up studies showing that the ingredients oxybenzone, octinoxate, octinoxate, octisalate, octocrylene, homosalate and avobenzone are all systematically absorbed into the body after a single use and the sunscreen ingredients could be detected on the skin and in blood weeks after application ceased. Maternal oxybenzone exposure also correlated with Hirschspring disease, a neo natal intestinal birth defect due to the failure of the Enteric Neural Crest cells to migrate to the hind gut during five to twelve weeks of embryogenesis which could be explained by oxybenzone inhibition of migration of specific muscle cells. Frequent application of sunscreen is recommended for sunburn protection but these studies indicate the damaging biological and environmental side effects.

Periodontal disease is a chronic inflammatory disorder of the gums variously referred to as gum disease, periodontitis, and gingivitis. Since the time that fluoride came into widespread use in drinking water and toothpaste to help reduce tooth loss due to decay, gum disease has become the largest cause of tooth loss in the adult population of the United States, accounting for approximately 70% of such losses. The disorder results from the accumulation of plaque, particularly within the gum line, which, unless effectively removed, produces a chronic inflammatory process of the gingiva that spreads and destroys the tissues supporting the tooth as well as the tooth itself, Effective removal of plaque is difficult, even with a vigorous and sustained program of brushing and flossing, and it has become clear that for effective control of periodontal disease, a more specific treating agent is needed.

### SUMMARY

Disclosed herein are compositions comprising: at least 0.1% (w/v) cannabidiol (CBD); at least 10% (w/v) ascorbic acid; approximately 10% to 25% (w/v) glucosamine; and water, wherein the composition has a pH of about 3.5 to about 4.1.

Also disclosed herein are compositions comprising: at least 0.1% by weight cannabidiol (CBD), or a pharmaceutically acceptable salt thereof 20-80% by weight calcium source; 10-40% by weight ascorbic acid, or a pharmaceutically acceptable salt thereof 5-20% by of a precursor or stimulant of epinephrine or nor-epinephrine production selected from the group consisting of tyrosine and phenylalanine; and 10-40% by weight anti-inflammatory substance.

Further disclosed herein are methods of treating diseases or disorders comprising applying a therapeutically effective amount of the composition described herein. In some embodiments, the disease or disorder is a skin disease or disorder, comprising rosacea, acne, inflammation, or a combination thereof. In some embodiments, the disease or disorder is a periodontal disease.

### DETAILED DESCRIPTION

The present disclosure provides stable, effective compositions that include CBD and ascorbic acid. The concentration of active ascorbic acid that is available to be delivered is maintained at a high concentration, while at the same time lowering the irritating effects commonly associated with compositions having a high concentration of organic acid. By providing, for example, a portion of the total ascorbic acid of the composition in the ascorbate salt form, the composition disclosed herein decreases the overall irritant nature of the solution without losing efficacy or desired biological effect. In addition, the compositions disclosed herein do not expand or lose integrity on storage. The present compositions are also far less likely to oxidize to yield an off color (e.g., to become darker or brown).

The present ascorbic acid-based compositions are effective for topical application to reduce epidermal wrinkling, such as that resulting from intrinsic aging or photo damage. For example, applying the present compositions within about six hours to skin that has received excess sun damage can attenuate the effects due to UV exposure and decrease sunburn and cell damage. The composition also brightens, soothes and helps hydrate skin.

In addition to treating aged or damaged skin, the present invention further includes compositions and methods for treating skin diseases and disorders such as inflammatory rosacea. The present invention provides formulations of ascorbic acid in combination with an anti-inflammatory agent such as, for example, glucosamine or other suitable aminosugar. Such combination formulations demonstrate efficacious treatment of skin diseases and disorders such as, for example, acne rosacea, in comparison to formulations of ascorbic acid without, for example, glucosamine.

Conventional treatment for acne rosacea includes cortisone therapy that involves continual use of cortisone, which causes connective tissue thinning. Conventional treatment for non-inflammatory rosacea (for example, red face due to surface blood vessels that become even more prominent after exposure to the sun or to the cold) includes metronizole, which is postulated to be an anti-inflammatory agent. Metronizole therapy requires continual use of metronizole while treatment is desired or necessary. Subjects that use cortisone or metronizole, which both require continual use, typically suffer rebound or recurrence of their inflammation after discontinuing use of either agent. The present invention provides a treatment for inflammatory rosacea that does not have the undesirable effects of cortisone and that is effective for individuals that are resistant to cortisone treatment. The present invention provides the further benefit of decreasing or eliminating rebound of acne rosacea after discontinuance of use.

Furthermore, the composition can be used for the treatment of acne. The composition not only treats inflammation but also has the ability to dry up sebum thereby reducing the growth of bacteria, e.g., P. acnes, and protect ethnic skin during treatment from the development of post inflammatory hyperpigmentation. Moreover, this new formulation would not require concomitant use of a sunscreen because the anti-inflammatory effects of the components would be able to prevent free radical damage in the skin and promote natural healing.

The ascorbic acid-based compositions disclosed herein are also effective in the treatment of periodontal disease, either to decrease inflammation prior to surgery or, in some cases, to even replace surgery.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Unless otherwise defined herein, scientific and technical term used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “cannabidiol (CBD)” refers hereinafter to one of at least 85 active cannabinoids identified in cannabis saliva. It is a 21-carbon terpenophenolic compound which is formed following decarboxylation from a cannabidiolic acid precursor. CBD can be isolated from cannabis or be produced synthetically. Cannabidiol is a major phytocannabinoid, accounting for up to 40% of the plant's extract. Cannabidiol has a very low affinity for CB1 and CB2 receptors but acts as an indirect antagonist of their agonists. CBD may potentiate THCs effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “tetrahydrocannabinol (THC)” refers hereinafter to the principal psychoactive constituent (or cannabinoid) of the cannabis plant, although a minor component of C. saliva. THC has a partial agonist activity at the cannabinoid receptor CB1, and the CB2 receptor.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the central and peripheral nervous system, but also in the lungs, liver and kidneys. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC. The CB2 receptor is expressed mainly in the immune system and in hematopoietic cells. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids) The principal endogenous ligand for the CB2 receptor is 2-arachidonylglycerol (2-AG).

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids.

The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

### 2. Compositions

a. Compositions for Treating Skin Disorders

Disclosed herein is a composition comprising: at least 0.1% (w/v) cannabidiol (CBD), or a pharmaceutically acceptable salt thereof; at least 10% (w/v) by weight ascorbic acid, or a pharmaceutically acceptable salt thereof; approximately 10-25% (w/v) glucosamine, or a pharmaceutically acceptable salt thereof; and water. The composition has a pH of about 3.5 to about 4.1.

Because of the potential problems of skin irritation with formulations containing high concentrations of ascorbic acid, it is generally advantageous to adjust the pH of such formulations to at least about 3.5. To achieve an optimum combination of low irritability and high stability, the present compositions are typically formulated to have a pH of about 3.7 to about 4.1 and, for example, between about 3.8 to about 4.0.

The “high pH” formulations of the present compositions are less irritating than high concentrations of L-ascorbic acid (with its inherent low pH, e.g., circa 2.0-2.5) because the relatively higher pH avoids the skin irritation problem often encountered with harsh chemical peels or solutions with pH values below 3.5. The present compositions are also very stable on short- and long-term storage, while maintaining a high degree of effectiveness.

The composition may comprise at least 0.1% (w/v) CBD, or a pharmaceutically acceptable salt thereof. In some embodiments, the composition comprises between about 0.1-10% (w/v) CBD. In certain embodiments, the composition comprises between about 0.1-1% (w/v) CBD. In exemplary embodiments, the composition comprises between 0.2% and 0.5% (w/v) CBD.

The composition may comprise at least 10% (w/v) by weight ascorbic acid, or a pharmaceutically acceptable salt thereof. In some embodiments, the composition comprises about 15% (w/v) ascorbic acid. Herein, the amount of ascorbic acid present in a composition refers to the total amount of ascorbic acid and ascorbate present stated as if all was present in the acid form.

The present compositions may also include a compound which can function as an anti-inflammatory agent. Examples of suitable anti-inflammatory agents include anti-inflammatory sulfur-containing compounds and anti-inflammatory aminosugars. The sulfur-containing anti-inflammatory compound is typically a sulfur containing amino acid or related derivative such as cystine, cysteine, N-acetyl cysteine, glutathione, cysteamine, S-methylcysteine, methionine and the like. Examples of suitable anti-inflammatory aminosugars include glucosamine, mannosamine, N-acetylmannosamine, galactosamine, glucosamine-6-phosphate, N acetylglucosamine, N-acetylmannosamine, N-acetylgalactoseamine and the like. The composition may comprise approximately 1-25% (w/v) glucosamine, or a pharmaceutically acceptable salt thereof.

The present compositions generally also include a non-toxic zinc salt, such as zinc sulfate. The zinc salt is generally present in about 0.5 to about 5.0% (w/v). Very effective results can typically be obtained with compositions that include no more than about 3.0% (w/v) zinc salt. For example, a number of present compositions are commonly formulated with about 0.5 to about 2.0% (w/v) zinc sulfate together with the other components described herein.

The composition of the present invention may further include one or more compounds capable of serving as a stimulant of protein synthesis and/or precursor to melanin synthesis. This component is generally present in about 1 to about 10% (w/v), and, for example, between about 3 to about 8% (w/v), based on the total composition. Typically, this component includes a tyrosine compound. As employed herein, a “tyrosine compound” is tyrosine or a compound that is capable of generating tyrosine upon chemical and/or biological transformation. Examples of suitable tyrosine compounds for use in the present compositions include tyrosine, N-acetyltyrosine, tyrosine ethyl ester hydrochloride, and tyrosine phosphate.

In some embodiments, the composition may further comprise at least one plant extract or oil. The plant extracts or oils include limonene, echinacea, magnolia bark, bisabol, myrcene, copaiba, lavender, cedarwood, mastic, other cannabis terpenes, or any combination thereof.

The composition may be a water soluble emulsion, such that hydrophobic CBD is incorporated with hydrophilic ascorbic acid. See for example, US20180360704, incorporated herein by reference.

The composition made be made by any method known in the art. In some embodiments, the method of making the compositions disclosed herein comprises, (a) dissolving about 10% to about 50% of the ascorbic acid in water at a temperature of between about 60° C. to about 90° C. to provide an aqueous ascorbic acid solution of at least 20% (w/v); (b) cooling the aqueous ascorbic acid solution to below about 40° C.; (c) combining the aqueous ascorbic acid solution with water, glucosamine, CBD and ascorbic acid to provide a composition comprising water, approximately 10% to 25% (w/v) glucosamine, at least 0.1% CBD, and at least 10% (w/v) ascorbic acid; and (d) adjusting the pH of the composition to about 3.5 to about 4.1.

It has been found that ascorbic acid-based topical formulations in which a substantial portion of the ascorbic acid has been pretreated in accordance with the present invention exhibit particularly good storage stability. As noted above, for the purposes of this application, “pretreated” ascorbic acid also refers to ascorbic acid that has been dissolved in water at a relatively high temperature to form a concentrated ascorbic acid solution. Typically, the ascorbic acid is dissolved in water at between about 60 to about 90° C. (e.g., between about 75 to about 80° C.) to form a concentrated solution that contains at least about 20% (w/v) ascorbic acid. The ascorbic acid is dissolved in water in the acid form, i.e., the resulting solution will have a relatively low pH (circa 2.0-2.5). After dissolution, the concentrate is generally heated for an additional period of time (e.g., 0.25 to 1.0 hour) and cooled to below about 40° C. before being incorporated into the final formulation. If the pretreated ascorbic acid concentrate is to be stored prior to formulation, it may be stored at room temperature or below (e.g., about 3 to about 20° C.) and/or under conditions that exclude oxygen-containing gases such as air (e.g., in a sealed container or blanketed with an inert gas such as argon or nitrogen). In the present compositions, at least about 10% of the ascorbic acid present may be pretreated ascorbic acid. Typically, no more than about 50% of the ascorbic acid present has been pretreated to obtain the enhanced stability properties of the compositions while minimizing the additional processing steps and costs associated with the pretreatment of the ascorbic acid.

The ascorbic acid and tyrosine compound components of the present compositions may be formulated in part or whole in a neutralized or salt form. Acceptable amine salts include the acid addition salts (e.g., formed with a free amino group of a tyrosine compound) and may be formed with inorganic acids such as, for example, hydrochloric or phosphoric acids, or such organic acids as acetic, oxalic, tartaric, mandelic, and the like, Salts formed with the free carboxyl groups may also be derived from inorganic bases such as, for example, sodium, potassium, ammonium, calcium, or ferric hydroxides, and such organic bases as isopropylamine, trimethylamine, histidine, procaine and the like. As noted elsewhere herein, since the present compositions have a pH of about 3.5 or above (and typically at least about 3.7) the ascorbic acid is typically at least partially present in the form of ascorbate salt(s), or possibly as an equilibrium reaction between ascorbic acid and monohydroascorbic acid. Commonly, the pH of the composition is adjusted to the desired value by adding sufficient base, such as sodium hydroxide, potassium hydroxide and/or ammonium hydroxide, to achieve the desired value. In such situations, the ascorbate would exist at least in part in the form of sodium hydroxide, potassium and/or ammonium ascorbate.

The water used for preparing the compositions of the present invention may be distilled and/or deionized, but any water may be used that does not contain contaminants that would affect the stability of the ascorbic acid present in the composition. For example, the presence of certain metal ions such as copper and iron salts, is known to affect the stability of ascorbic acid. The effects of water of varying purity on ascorbic acid stability is discussed in Meucci, et al., “Ascorbic Acid Stability in Aqueous Solutions,” Acta Vitaminol. Enzymol. 7(34): 147-54 (1985), the disclosure of which is incorporated herein by reference.

The compositions may be formulated for administration by, for example, topical formulations. Techniques and formulations may generally be found in “Remington's Pharmaceutical Sciences,” (Meade Publishing Co., Easton, Pa.), Therapeutic compositions must typically be sterile and stable under the conditions of manufacture and storage.

The disclosed compositions can be topically administered. Topical compositions that can be applied locally to the skin may be in any form including solids, solutions, oils, creams, ointments, gels, lotions, foundations, shampoos, leave-on and rinse-out hair conditioners, milks, cleansers, moisturizers, sprays, skin patches, and the like. Topical compositions include: a disclosed composition and a carrier. The carrier of the topical composition preferably aids penetration of the ingredients. The carrier may further include one or more optional components. The amount of the carrier employed in conjunction with a disclosed composition is sufficient to provide a practical quantity of composition for administration per unit dose.

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

Carriers for topical application which may be used with the present invention include, but are not limited to, alkyleneglycols, or alkyleneglycols in combination with one or more derivatives of hydroxyalkylcellulose. In one illustrative embodiment, the alkylene glycol is propyleneglycol and the hydroxyalkylcellulose is hydroxypmpylcellulose.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollients) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Other materials that may be used in conjunction with the present invention include: almond meal, alumina, aluminum oxide, aluminum silicate, apricot seed powder, attapulgite, barley flour, bismuth oxychloride, boron nitride, calcium carbonate, calcium phosphate, calcium pyrophosphate, calcium sulfate, cellulose, chalk, chitin, clay, corn cob meal, corn cob powder, corn flour, corn meal, corn starch, diatomaceous earth, dicalcium phosphate, dicalcium phosphate dihydrate, fullers earth, hydrated silica, hydroxyapatite, iron oxide, jojoba seed powder, kaolin, magnesium trisilicate, mica, microcrystalline cellulose, montmorillonite, oat bran, oat flour, oatmeal, peach pit powder, pecan shell powder, polybutylene, polyethylene, polyisobutylene, polymethylstyrene, polypropylene, polystyrene, polyurethane, nylon, teflon (i.e. polytetrafluoroethylene), polyhalogenated olefins, pumice rice bran, rye flour, sericite, silica, silk, sodium bicarbonate, sodium silicoaluminate, soy flour synthetic hectorite, talc, tin oxide, titanium dioxide, tricalcium phosphate, walnut shell powder, wheat bran, wheat flour, wheat starch, zirconium silicate, and mixtures thereof. Also useful are mixed polymers (e.g., copolymers terpolymers, etc.), such as polyethylene/polypropylene copolymer, polyethylene/propylene/isobutylene copolymer, polyethylene/styrene copolymer, and the like. Typically, the polymeric and mixed polymeric particles are treated via an oxidation process to destroy impurities and the like. The polymeric and mixed polymeric particles may also be crosslinked with a variety of common crosslinking agents. Examples of common cross-linking agents include: butadiene, benzene, methylenebisacrylamide, allyl ethers of sucrose, allyl ethers of pentaerythritol, and mixtures thereof. Other examples of useful particles include waxes and resins, such as: paraffins, carnauba wax, ozokerite wax, candelilla wax, urea-formaldehyde resins and the like. When waxes and resins are used it is important that these materials are solids at ambient and skin temperatures.

b. Compositions for Treating Periodontal Disease

Disclosed is a composition comprising: at least 0.1% by weight cannabidiol (CBD), or a pharmaceutically acceptable salt thereof; 20-80% by weight calcium source; 10-40% by weight ascorbic acid, or a pharmaceutically acceptable salt thereof; 5-20% by weight precursor or stimulant of epinephrine or nor-epinephrine production selected from the group consisting of tyrosine and phenylalanine; and 10-40% by weight anti-inflammatory substance.

The composition may comprise at least 0.1% by weight CBD, or a pharmaceutically acceptable salt thereof. In some embodiments, the composition comprises between about 0.1-10% CBD by weight. In certain embodiments, the composition comprises between about 0.1-1% by weight. In exemplary embodiments, the composition comprises between 0.2% and 0.5% by weight CBD.

The composition may comprise 20-80% by weight calcium source. In some embodiments, the composition comprises 30-60% by weight calcium source.

The calcium source may be chosen from any source well-known in the state of the art, including, but not limited to, commonly utilized calcium salts. In some embodiments, the calcium source is selected from the group consisting of bone meal, calcium gluconate, calcium carbonate, calcium phosphate, and dolomite. In certain embodiments, the calcium source is bone meal. The calcium source may serve as an agent for tooth regeneration, as a gentle cleansing agent, and/or as a filler.

The composition may comprise 10-40% by weight ascorbic acid. In some embodiments, the composition comprises 15-30% by weight ascorbic acid. Herein, the amount of ascorbic acid present in a composition refers to the total amount of ascorbic acid and ascorbate present stated as if all was present in the acid form.

The composition may comprise 5-20% by weight precursor or stimulant of epinephrine or nor-epinephrine production selected from the group consisting of tyrosine and phenylalanine. In some embodiments, the composition comprises 10-15% by weight of the precursor or stimulant. In certain embodiments, the precursor or stimulant is tyrosine. As a precursor of epinephrine, tyrosine is a preferred substance for use in the present invention, because it has been shown in tissue culture to promote proliferation of the type of cell (fibroblasts) which are involved in the healing of periodontal tissue. Activity of this sort is exhibited by both epinephrine and nor-epinephrine, and consequently by precursors and stimulants of epinephrine and nor-epinephrine synthesis.

The composition may comprise 10-40% weight anti-inflammatory substance. In some embodiments, the composition comprises 15-30% by weight anti-inflammatory, substance.

In some embodiments, anti-inflammatory substance being selected from the group consisting of mannose, 2-deoxy-D-glucose, glucosamine, glucosamine-6-phosphate, N-acetylglucosamine, galactosamine, cysteine, glutamine, alanine. L-tryptophan, valine and creatinine.

In some embodiments, the anti-inflammatory substance is cysteine. Cysteine is preferred because it is bactericidal against Streptococcus mutans in addition to being anti-inflammatory in action. Other suitable amino acids include creatine, creatinine, L-tryptophan, valine, alanine, glycine, glutamine, aspartic acid, and S-methylcysteine, as well as the esters. N-benzenesulfonyl derivatives, and diazomethyl ketone and chloromethyl ketone analogs of the N-tosyl derivatives thereof, and the like.

In some embodiments, the anti-inflammatory substance is glucosamine. In certain embodiments; the glucosamine is in the form of a salt with a biocompatible acid. The biocompatible acid may be hydrochloric, sulfuric, phosphoric, or other biocompatible acid, as known in the art.

Other sugars and sugar derivatives having similar anti-inflammatory effects include 2-deoxy-D-glucose, 2-deoxy-D-galactose, mannose, D-mannosamine, D-galactosamine, and the like. Also useful are glucosamine-6-phosphate, N-acetyl-D-glucosamine, N-acetyl-D-galactosamine, uridine diphosphate (UDP) glucose, UDP-N-acetylglucosamine, and the like.

In addition to the above ingredients, which are directed toward the primary purpose of the present invention, the novel composition may include certain optional ingredients, such as fluoride ion sources, sudsing agents, flavoring agents, sweeting agents, anticalculus agents, antiplaque agents, coloring agents, opacifying agents, and the like, as described in U.S. Pat. No. 4,254,101, which is incorporated herein by reference.

The compositions may be formulated for administration by any of the known methods in the art. Techniques and formulations may generally be found in “Remington's Pharmaceutical Sciences,” (Meade Publishing Co., Easton, Pa.). Therapeutic compositions must typically be sterile and stable under the conditions of manufacture and storage.

The composition of the invention can be formulated as a toothpaste or a gel by milling in a conventional manner with an appropriate amount of glycerol, sorbitol, and water, plus a thickening agent (for example, xanthan gum) to produce the desired consistency, plus an opacifying agent if the toothpaste form is desired. These formulations are used in a conventional manner, with care to work the material into the gingival-tooth junction.

The route by which the disclosed compositions are administered and the form of the composition will dictate the type of carrier to be used. The composition may be in a variety of forms, suitable, for example, for systemic administration or topical administration.

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose; dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia. Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592: Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

The selection of ingredients in the carrier for oral compositions depends on secondary considerations like taste, cost, and shelf stability, which are not critical for the purposes of this invention.

The composition may be applied as solutions, creams, ointments, gels, sprays, and the like. Tice carrier may further include one or more optional components. The amount of the carrier employed in conjunction with a disclosed composition is sufficient to provide a practical quantity of composition for administration.

A carrier may include a single ingredient or a combination of two or more ingredients. Suitable carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a composition may further include one or more ingredients selected from emollients, propellants, solvents, humectants, thickeners, powders, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

### 3. Methods of Treatment

a) Skin Disorders

Disclosed herein is a method of treating skin disease or disorder comprising topically applying a therapeutically effective amount of the composition described herein to the affected skin. The skin disease or disorder may comprise acne rosacea, acne, inflammation, or a combination thereof. The composition may prevent, lessen or reduce the severity of post inflammatory hyperpigmentation, keloid formation or hypertrophic scarring. In some embodiments, the skin disease or disorder comprises a sun burn.

The compositions disclosed herein may be administered alone or in combination with a therapeutically effective amount of at least one additional therapeutic agents, antibiotics, analgesics, anti-allergenics and the like, or therapeutic regimen. The additional therapeutic agent(s) or regimens may be administered simultaneously or sequentially with the disclosed compositions. Sequential administration includes administration before or after the disclosed compositions. In some embodiments, the additional therapeutic agent or agents may be administered in the same composition. In other embodiments, there may be an interval of time between administration of the additional therapeutic agent and the disclosed composition. In some embodiments, administration of an additional therapeutic agent with a disclosed composition may allow lower doses of the other therapeutic agents and/or administration at less frequent intervals. When used in combination with one or more other active ingredients, the compositions of the present invention and the other active ingredients may be used in lower doses than when each is used singly.

For optimum efficacy, treatment in accordance with the presented method should be initiated as early as possible following exposure to sunlight or another radiation source or upon the occurrence of a rosacea acne outbreak, rash, dermatitis, adult acne or other inflammatory skin response.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

b) Periodontal Disease

Also disclosed herein is a method of treating skin disease or disorder comprising topically applying to the gingival-tooth junction a therapeutically effective amount of the composition described herein.

In some embodiments, the composition is applied by brushing it into the gingival-tooth junction. The composition of the present invention, when prepared in the form of a powder, can be used by lightly applying with a soft toothbrush into the gingival-tooth junction twice a day, followed, if desired, by rinsing. It can also be used in the form of a paste or gel, in which the total concentration can be as low as around 5% by weight. For use in treating periodontal disease, the powder form is advantageously used, without dilution. A preventive formulation without glucosamine can be used as a “matrix,” blown in with a periojet at the time of surgery to promote healing and also to provide a better attachment for tissue.

Periodontal disease is defined by at least one periodontal site with 3 millimeters or more of attachment loss and 4 millimeters or more of pocket depth. Moderate periodontal disease is defined as having at least two teeth with interproximal attachment loss of 4 millimeters or more or at least two teeth with 5 millimeters or more of pocket depth at interproximal sites. Severe periodontal disease is defined as having at least two teeth with interproximal attachment loss of 6 millimeters or more AND at least one tooth with 5 millimeters or more of pocket depth at interproximal sites. The composition may slow or prevent progression to moderate or severe periodontal disease or decrease attachment loss and pocket depths of any level of periodontal disease.

### 4. EXAMPLES

### Example

Moderate to Severe Acne and Acne Rosacea Treatment

Compositions of the present invention with 0.5% salicylic acid can be used to determine the effectiveness of the composition compared to a control treatment with 0.5% salicylic acid alone. The race and/or ethnicities (e.g. Caucasian, African American, Asian, Latino) of the subjects can be controlled to include a variety of skin tones and genetic background. The compositions and control is administered for 6 weeks.

Before the first administration clinical staff evaluate the clinical picture, family history, list of current medications, date of acne onset, past therapies for acne and their information. Evaluation of current acne includes identification and number of lesions, number of comedones and pustules, presence of redness in affected areas and assessment of size of affected areas on cheeks, forehead, chin and elsewhere if needed (chest or shoulders).

Subsequent to the first administration and during the 6 weeks of treatment, the subject is monitored by a clinician to assess for irritation, side effects, and proper use of product, as well as monitor the improvement in the acne using the same criteria as in the initial evaluation.

### Example 2

Treatment of Sunburn

As described in U.S. Pat. No. 6,217,914, incorporated herein by reference, a composition comprising ascorbic acid was shown to prevent a blistering sunburn when applied to facial skin of a Caucasian female immediately after exposure to desert sun from 3:00 to 4:30 pm, although the other three members of the group all had severe sunburn on their faces the following day.

A applying an ascorbic acid composition with CBD on the unprotected red (slightly inflamed) shoulders of two adult Caucasian women exposed more than two hours to intense afternoon sun produced a similar result in which the following day, the affected regions were replaced by a light tan, with no evidence of sunburn even though the test formulation was applied four hours after the unintended excessive sun exposure.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof

What is claimed is:

1. A composition comprising:
at least 0.1% (w/v) cannabidiol (CBD);
at least 10% (w/v) ascorbic acid;
approximately 10% to 25% (w/v) glucosamine; and
water,
wherein the composition has a pH of about 3.5 to about 4.1.

2. The composition of claim 1, wherein the CBD is present in an amount of about 0.1-10% (w/v) of the composition.

3. The composition of claim 1 or claim 2, wherein the CBD is present in an amount of about 0.1-1% (w/v) of the composition.

4. The composition of any of claims 1-3, wherein the ascorbic acid is present in an amount of about 15% to about 25% (w/v) of the composition.

5. The composition of any of claims 1-4, wherein the ascorbic acid is present in an amount of about 15% (w/v) of the composition.

6. The composition of any of claims 1-5, wherein the water is distilled or deionized water.

7. The composition of any of claims 1-6, further comprising 0.5-5% (w/v) zinc salt.

8. The composition of any of claims 1-7, further comprising 1-10% (w/v) of a stimulant of protein synthesis and/or precursor to melanin synthesis.

9. The composition of claim 8, wherein the stimulant of protein synthesis and/or precursor to melanin synthesis is selected from the group consisting of tyrosine, N-acetyltyrosine, tyrosine ethyl ester hydrochloride, and tyrosine phosphate.

10. The composition of any of claims 1-9, further comprising at least one plant extract or oil selected from the group consisting of: limonene, echinacea, magnolia bark, bisabol, myrcene, copaiba, lavender, cedarwood, mastic, other cannabis terpene, or combinations thereof.

11. The compositions of any of claims 1-10, wherein the composition is a water soluble emulsion.

12. A method of making a composition of any of claims 1-11 comprising:
(a) dissolving about 10% to about 50% of the ascorbic acid in water at a temperature of between about 60° C. to about 90° C. to provide an aqueous ascorbic acid solution of at least 20% (w/v);
(h) cooling the aqueous ascorbic acid solution to below about 40° C.;
(c) combining the aqueous ascorbic acid solution with water, glucosamine, CBD and ascorbic acid to provide a composition comprising water, approximately 10% to 25% (w/v) glucosamine, at least 0.1% CBD, and, at least 10% (w/v) ascorbic acid and
(d) adjusting the pH of the composition to about 3.5 to about 4.1.

13. A method of treating a skin disease or disorder in a subject comprising topically applying a therapeutically effective amount of the composition of any one of claims 1-11 to the affected skin.

14. The method of claim 13, wherein the skin disease or disorder comprises acne rosacea, acne, inflammation, or a combination thereof.

15. The method of claim 13 or 14, wherein the skin disease or disorder comprises a sun burn.

16. A composition comprising
at least 0.1% by weight cannabidiol (CBD), or a pharmaceutically acceptable salt thereof;
20-80% by weight calcium source;
10-40% by weight ascorbic acid, or a pharmaceutically acceptable salt thereof;
5-20% by of a precursor or stimulant of epinephrine or nor-epinephrine production selected from the group consisting of tyrosine and phenylalanine; and
10-40% by weight anti-inflammatory substance.

17. The composition of claim 16, wherein the CBD is present in an amount of about 0.1-10% by weight of the composition.

18. The composition of claim 16 or claim 17, wherein the CBD is present in an amount of about 0.1-1% by weight of the composition.

19. The composition of any of claims 16-18, wherein the calcium source is present amount of 30-60% by weight.

20. The composition of any of claims 16-19, wherein the calcium source is selected from the group consisting of bone meal, calcium gluconate, calcium carbonate, calcium phosphate, and dolomite.

21. The composition of any of claims 16-20, wherein the calcium source is bone meal.

22. The composition of any of claims 16-21, wherein the ascorbic acid is present in an amount of 15-30% by weight.

23. The composition of any of claims 16-22, wherein the precursor or stimulant is present in an amount of 10-15% by weight.

24. The composition of any of claims 16-23, wherein the precursor or stimulant is tyrosine.

25. The composition of any of claims 16-24, wherein the anti-inflammatory substance is present in an amount of 15-30% by weight.

26. The composition of any of claims 16-25, wherein the anti-inflammatory substance being selected from the group consisting of mannose, 2-deoxy-D-glucose, glucosamine, glucosamine-6-phosphate, N-acetylglucosamine, galactosamine, cysteine, glutamine, alanine, L-tryptophan, valine and creatinine.

27. The composition of any of claims 16-26, wherein the anti-inflammatory substance is glucosamine.

28. The composition of any of claims 16-27, wherein the glucosamine is in the form of a salt with a biocompatible acid.

29. The composition of any of claims 16-26, wherein the anti-inflammatory substance is cysteine.

30. A method for preventing or treating periodontal disease in a subject, comprising applying to the gingival-tooth junction a therapeutically effective amount of a composition of any of claims 16-29.

31. The method of claim 30, wherein the composition is applied by brushing it into the gingival-tooth junction.
